(19) United States
(12) Patent Application Publication
(10) Pub. No.: US 2007/0203507 A1
(43) Pub. Date: Aug. 30, 2007

(54) Suturing apparatus and methods

(75) Inventors: Glen W. McLaughlin, San Carlos, CA (US); Imraan Aziz, Oakland, CA (US); Ron Songer, Palo Alto, CA (US); Jeffrey Christian, Morgan Hill, CA (US); Anil Singhal, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: G-Surge Medical Solutions, Inc., Menlo Park, CA (US)

(21) Appl. No.: 11/511,873

(22) Filed: Aug. 28, 2006

Related U.S. Application Data
(60) Provisional application No. 60/711,857, filed on Aug. 26, 2005.

(51) Int. Cl. A61B 17/04

(52) U.S. Cl. 606/144

(57) ABSTRACT

Embodiments of the invention provide methods and apparatus for suturing tissue penetrations made during minimally invasive surgery. One embodiment of an apparatus for suturing tissue penetrations comprises a shaft, suture capture surface coupled to the shaft, and at least one pair of needles or other penetrating members advanceable from the shaft. The shaft can be detachably coupled to a hand-piece. The surface has a deployed and a non-deployed configuration and is configured to capture a suture in the deployed configuration and retain it in the non-deployed configuration. The surface can be deployed by a frame or other expandable structure. The penetrating members are configured to be coupled to a suture and are advanceable from the shaft by an advancement member or other means to deliver a suture end portion to the deployed surface. Sutures can be contained in a replaceable cartridge detachably coupled to the apparatus.

### CROSS-REFERENCES TO RELATED APPLICATIONS

This application claims the benefit of priority of U.S. Provisional Application Ser. No. 60/711,857 (Attorney Docket No. 025861-000100US), filed on Aug. 26, 2005, the full disclosure of which is incorporated herein by reference.

### BACKGROUND OF THE INVENTION

### Field of the Invention

Embodiments of the invention relate generally to medical devices and methods. More particularly, embodiments of the invention relate to methods and apparatus for suturing tissue penetrations, such as those formed during laparoscopic procedures.

Trocar devices have been used to place access ports for laparoscopic surgical procedures for many years. The access ports typically include cannulas which can have diameters from 5 mm all the way up to over 35 mm. The trocar device has a sharpened tip which produces a tissue penetration, and the cannula (which typically is disposed coaxially over the trocar) is left behind to provide the access port for performing laparoscopic procedures. Most cannulas have one way valves within a central lumen so that pneumoperitoneum (i.e. the pressurization of the abdomen) can be maintained during the procedure. After the surgeon has finished with the procedure, the cannula is removed exposing the penetration (defect) created from the trocar device. The surgeon has several methods of choice of how to seal the defect.

The first approach and simplest is to do nothing. For the smaller access ports with diameters of about 5 mm there are few consequences. The defect usually heals with no complications. With penetration defects above 5 mm, however, there is a substantial risk of complications if they are not closed in some manner.

Hand suturing the presenting tissue layers together is another approach, but this method has several limitations. First, it is highly dependent on the overall dexterity and skill of the responsible surgeon. Next, in order to perform the closure, the pneumoperitoneum needs to be compromised, heightening the risk of herniation during the suturing procedure as well as placing the suture within the bowel during closure. Defect closures tend to be inconsistent and time intensive to perform. In most cases, some form of visualization from within the abdominal cavity needs to be used to assist the surgeon in safe and effective suture placement. This makes it difficult to accurately close the last access port and often requires a second surgeon. Thus, there is a need for systems that allow the surgeon to produce timely, consistent, and accurate closures while requiring minimal skill or dexterity.

### BRIEF SUMMARY OF THE INVENTION

Embodiments of the invention provide methods and apparatus for suturing tissue penetrations, particularly percutaneous penetrations made for access during minimally invasive surgical procedures, such as laparoscopic procedures, thoracoscopic procedures, and the like. Embodiments of the invention are particularly useful for closing such percutaneous penetrations, including those that are larger than about 4 to 5 mm, typically larger than about 10 mm, and often about 20 mm or larger. Various embodiments can utilize subdermal deployment of needles or other penetrating members for advancing a suture to close the penetrations at a sub-dermal level. Such embodiments can be configured to allow the surgeon to produce timely, consistent, accurate, and reliable closures with minimal risk of reopening of the penetration site or other failures. Further, such embodiments can be configured to be relatively simple to operate without requiring advanced skill or dexterity on the part of the surgeon. Ease of use is facilitated by the fact that intra-abdominal or other visualization or imaging is not required since, as will be explained herein, the apparatus can be configured to allow the surgeon to place the apparatus at the desired tissue site by feeling the mechanical engagement of the device with the abdominal wall (or other tissue wall).

In addition to ease of use, embodiments of the invention allow for reduced post operative complications (such as herniated penetration sites, infection), less tissue trauma from poorly closed defects, decreased operating times and faster wound healing and recovery times.

Various embodiments and methods of the invention comprise deploying a suture capture surface on a posterior region of tissue, at least partially circumscribing the penetration site. In various instances, the capture surface can circumscribe 30, 60 or substantially 100% of the tissue penetration site. At least one needle or other penetrating member is then advanced through the tissue to deliver a pair of suture ends and to the deployed suture capture surface. In preferred embodiments, at least one pair of needles or other tissue penetrating members are advanced through to deliver the pair of suture ends. The needles can be captured independent of their point of entry into the capture surface, or they can be directed at target capture zones in the capture surface described herein. The needle structures are then withdrawn, leaving the deployed suture ends captured by or within the suture capture surface. The capture surface is then withdrawn, typically through an interior region of the penetration, to bring the suture ends to an anterior side of the tissue, typically external to the patient.

The suture ends will usually be on a common length of suture, i.e. a continuous length having opposite ends which comprise the two ends. When using such a single length suture, the suture ends are drawn together to close the penetration, and the suture is fastened together to hold the penetration closed. Also preferably, the suture ends are advanced using a pair of penetrating members, however, this can also be done using a single penetrating member which is advanced into a first location on the capture surface and then subsequently a second location, for example by rotating the surface.

In some instances, the suture ends may be on two separate lengths of suture. In those cases, the two lengths of suture will usually be attached together prior to drawing the two attached lengths together to close the penetration. Alternatively, the two separate lengths may be attached together and then exchanged for a single continuous length of suture. The exchanged single length of suture may then be drawn together to close the penetration. The final suture or pair of suture lengths will usually be fastened together, typically by tying, to hold the penetration closed.

The suture capture surface will typically be “deployable.” That is, the suture capture surface will have a low profile or reduced diameter configuration which permits it to be introduced through the tissue penetration site. Deployment then comprises radially expanding the capture surface on the posterior region of the tissue to form a needle target region or regions. The suture capture surface may have an annular geometry which is generally symmetric about a shaft which is used for introduction. Alternatively, the suture capture surface may be non-annular and may comprise a pair of discrete target regions disposed symmetrically on either side of the deployment shaft. Still further alternatively, the suture capture surface could have a non-annular, non-symmetric geometry.

Embodiment of methods and apparatus of the invention are applicable to closure of tissue penetration sites in a number of locations throughout the human body including the abdominal wall, the thoracic wall and other locations in the chest wall. Closures can also be performed for tissue penetrations into organs such as the heart, lung, intestine and other organs. Also, sutures can be positioned to perform a closure in a particular layer in the tissue penetration site such as a dermal, muscular, adipose, cartilage or fascial layer. Positioning in a particular layer can be accomplished using depth positioning means described herein. In preferred embodiments, the entire suture path can be positioned sub-dermally with a portion of the suture path being close to or at the peritoneum or other body cavity surface layer so as to prevent or impede tissue herneation into the tissue penetration site. The suture path can be angled or curved with the suture entry point being sub-dermally positioned and the exit point being through the peritoneum or other tissue cavity surface layer.

One embodiment of an apparatus for performing a closure of the tissue penetration comprises a shaft having a distal end and a lumen, a suture capture surface coupled to the shaft, at least one pair of penetrating members advanceable from the shaft. The shaft will typically be configured to be detachably coupled to a hand-piece. The capture surface has a deployed configuration and a non-deployed configuration. The surface is configured to capture a suture in the deployed configuration and retain the suture in the non-deployed configuration. The penetrating members are configured to be coupled to a suture and are advanceable from the shaft to deliver an end portion of the suture to the suture capture surface when the surface is in the deployed configuration. The penetrating member can comprise a needle, an anchoring member or an anchoring needle. The penetrating members can be advanced from the shaft by means of an advancement member or other mechanical linkage which can be coupled to a mechanism in the hand piece. The penetrating member can also include a movable tip portion, such as a pivotal portion, which re-orients upon entry into the suture capture surface to anchor the penetrating member into the surface.

The capture surface will typically have an annular geometry which is generally symmetric about the shaft, though non-annular geometries and non symmetric configurations are also contemplated. Other geometries can include inwardly conical (relative to the proximal portion of the shaft), mushroom shaped, rectangular, triangular and like shapes. The surface is penetrable to allow penetration by the needle or other penetrating member and is also configured to capture the suture in some manner. For example, the suture may carry a barb or other element which can pass through the surface but which will be trapped by the surface to prevent withdrawal of the suture when the needle is retracted. The surface will typically comprise a conformable material such as a mesh or elastic membrane such as silicone, polyurethane or other elastomer known in the art. The surface can be configured to capture the penetrating member and the suture independent of the point of entry into the surface. The surface can also include at least one target zone configured to align with and capture an advanced penetrating member. In one embodiment, at least two target zones can be symmetrically disposed on either side of the shaft. The target zones can have a different thickness or material from a remainder portion of the capture surface. In a preferred embodiment, the radial cross section of the surface can have a convoluted shape which allows for ease of packing of the surface in the non-deployed state and a larger surface area in the deployed state with less required deployment force. The surface can deployed by hydraulic or pneumatic means and in preferred embodiments, is expanded by a mechanical expansion using an expandable frame described below. The surface can also be expanded by other expandable structures such as an expandable balloon, foam support, spring or other shape memory structure. The balloon structure can be puncture resistant and/or self sealing to resist puncture by the advancing penetrating members.

In many embodiments, the suture capture surface will be mounted over an expandable frame which can be shifted between a low profile radially constrained configuration and a radially expanded configuration so as to expand the capture surface to its deployed configuration. The frame typically will provide for expanding and contracting the capture surface by advancing and/or withdrawing a mechanical linkage which can be coupled to a mechanism in the hand-piece. Alternatively, the frame may be expanded by an expandable balloon or other expandable structure or the frame may be self-expanding so that it will expand when it is released from a constraining receptacle in or on an advancement shaft (e.g. a lumen in the shaft).

In many embodiments, the apparatus will include a suture cartridge which is integral to or otherwise coupled to the shaft. The proximal end of the cartridge can be configured to be detachably coupled with a hand-piece either directly or via a shaft extending from the hand-piece. The distal end of the cartridge will typically be coupled to a proximal end of the expandable frame structure. The cartridge can be configured to hold at least one suture and a tissue penetrating member. Each suture will have at least one needle or other tissue penetrating member coupled to an end of the suture. Together, they comprise a suture assembly. In various embodiments, the cartridge can hold at least one, two, or three suture assemblies or any other selected number. The cartridge is configured to be engaged by at least one advancement member, such as a push rod for advancing the penetrating members into tissue. The push rod can be mechanically linked to a mechanism in the hand-piece or shaft. Pneumatic and hydraulic penetrating member advancement means are also contemplated. The cartridge can be packed with sutures having tissue penetrating members on both ends, such that each pair of suture ends advanced into tissue share a common length of suture, or alternatively, it may be packed with sutures having a needle only on one end, such that the pair of suture ends are on separate sutures. The cartridge can also have a bar code or other identifying indicia identifying one or more characteristics of the sutures (e.g., type (e.g., PROLINE) length, needle type, needle on both ends, number, etc.)

Embodiments of methods and apparatus of the invention are applicable to closure of tissue penetration sites in a number of locations throughout the body including the abdominal wall, the thoracic wall and other locations in the chest wall. Closures can also be performed for tissue penetrations into organs such as the heart, lung, intestine and other organs. Also, sutures can be positioned to perform a closure in a particular layer in the tissue penetration site such as a dermal, muscular, adipose, cartilage or fascial layer. Positioning in a particular layer can be accomplished using depth positioning means described herein. In preferred embodiments, the entire suture path can be positioned sub-dermally with a portion of the path close to or at the peritoneum or other body cavity surface layer so as to prevent or impede tissue herneation into the tissue penetration site. The tissue path can be angled or curved with the suture entry point being sub-dermally positioned and the exit point being through the peritoneum or other cavity surface layer. Additional aspects and embodiments of the invention are described in more detail below.

### BRIEF DESCRIPTION OF THE DRAWINGS

FIG. 1 is a perspective view illustrating an embodiment of the closure apparatus.

FIG. 1a is a perspective view illustrating the distal portion of the embodiment of FIG. 1 showing the suture capture surface in the deployed state.

FIG. 1b is a perspective view illustrating the distal portion of the embodiment of FIG. 1 showing the deployment of the tissue penetrating members.

FIG. 1c is a cut away view illustrating the distal portion of the embodiment of FIG. 1 showing the expandable frame.

FIG. 1d is a cut away view illustrating the proximal portion of the embodiment of FIG. 1 showing an attached hand-piece and mechanism for deployment of the expandable frame and penetrating members.

FIG. 1e is a lateral view illustrating the modular construction of an embodiment of the closure apparatus.

FIG. 1f is cross sectional view along lines A-A in FIG. 1.

FIG. 1g is cross sectional view along lines B-B in FIG. 1.

FIG. 1h is cross sectional view along lines C-C in FIG. 1.

FIG. 1i is a cut away view illustrating an embodiment of the closure apparatus structure having an expandable balloon for expansion of the frame structure.

FIG. 1j is a cut away view illustrating an embodiment of the closure apparatus having an expandable foam support.

FIG. 2a is a phantom view illustrating an embodiment of the suture capture member in the non-deployed state as well as the suture cartridge with non-deployed tissue penetrating members.

FIG. 2b is a phantom view illustrating an embodiment of the suture capture member in the deployed state as well as the suture cartridge.

FIG. 3 is a lateral view illustrating the suture capture member in the deployed state with the penetrating members being advanced by push rods through various tissue layers at the tissue penetration site.

FIG. 4 is a perspective view illustrating another embodiment of the suture capture member having annular framing. The penetrating members are being advanced by the push rods into the surface of the suture capture member.

FIG. 5 is a perspective view of the embodiment of FIG. 4 showing the penetrating members after they have pierced the elastomeric membrane and the push rods are being retracted

FIG. 6 is a perspective view of the embodiment of FIG. 4 showing the suture capture member in the collapsed/non-deployed state after the anchors have been deployed.

FIG. 7 is a perspective view illustrating another embodiment of suture capture member having a conical shaped suture capture surface with an anchor deployed through the surface.

FIG. 8 is perspective/cut away view illustrating an embodiment of a suture capture member having a scaffolding structure that creates a four point frame for the suture capture surface.

FIG. 9 is a perspective view illustrating an embodiment of a suture capture member having an annular plate shaped surface with target zones for the penetrating members to be set.

FIG. 10 is a perspective view illustrating an embodiment of a suture capture having a mushroom shape with stalk portion configured to pre-tension the tissue layer prior to suture placement for increased consistency of suture placement.

FIGS. 11a and 11b are cut away views of the abdominal cavity illustrating the tissue penetration site in different body types with different fat content and body mass index. The fascia, muscle, and peritoneum layers remain relatively constant while the fat layer on top of the fascia can have a substantial amount of variation.

FIGS. 12a and 12b are cut away views illustrating the final placement of the sutures positioned in the body types shown in FIGS. 11a and 11b. They show how embodiments of the closure apparatus can be used to place sutures in at a desired location in a tissue penetration site independent of a patient's body mass index.

FIG. 13 is a cut away view of the abdominal cavity illustrating the placement and final configuration of a suture deployed by the closure apparatus to close the tissue penetration site.

FIGS. 14a-14h are a series of lateral views illustrating the deployment of a suture at the tissue penetration site using the closure apparatus and the subsequent manipulation of the suture to close the penetration site.

FIG. 15 is a perspective view of an embodiment of a capture surface having a convoluted cross-sectional shape in the non-deployed state.

FIG. 16 is a lateral view of an embodiment of a capture surface having a convoluted cross-sectional shape in the non-deployed state.

FIGS. 17a-17d are cross sectional views of the embodiment of FIG. 16 along lines A-A, B-B, C-C and D-D.

FIGS. 18a-18d are lateral view illustrating different embodiments of penetrating members.

FIGS. 19a-19e are lateral views illustrating use of a self orienting penetrating member/anchor to capture an attached suture in the capture surface.

FIG. 20 is a lateral view illustrating an embodiment of the closure apparatus configured to deliver and suture a prosthetic membrane/structure at a tissue penetration site.

### DETAILED DESCRIPTION OF THE INVENTION

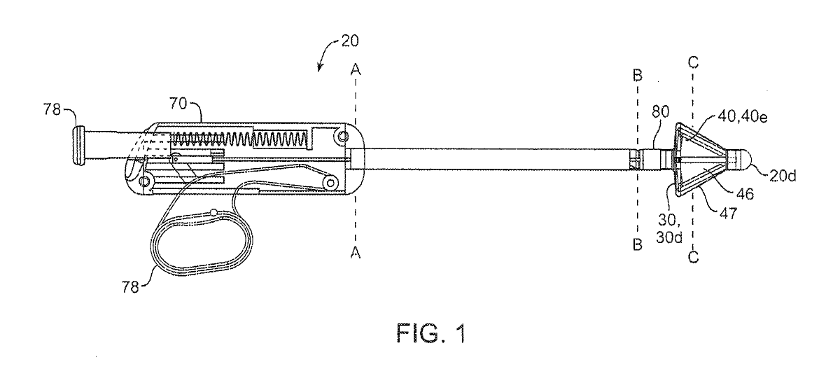

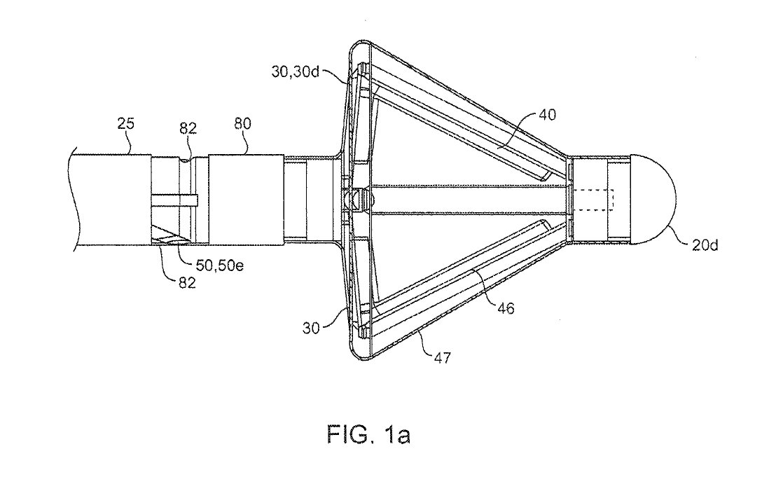

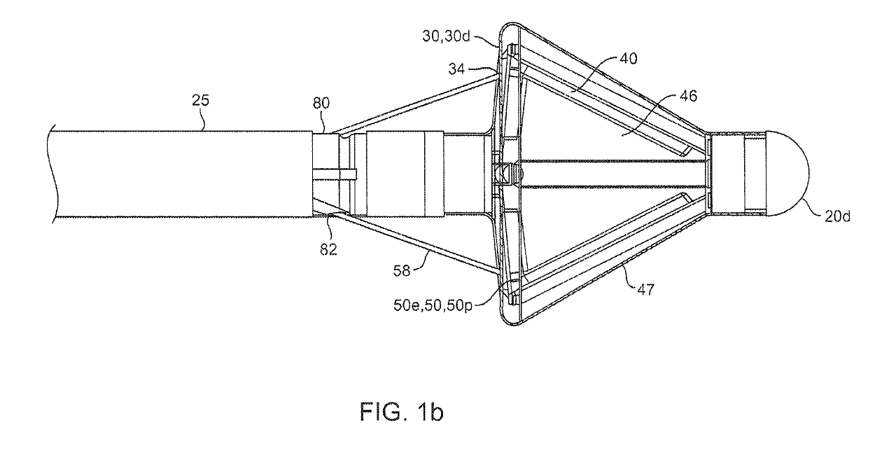

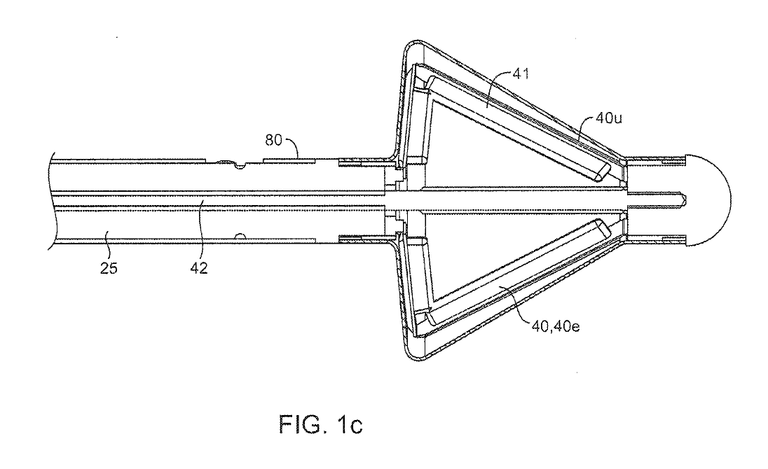

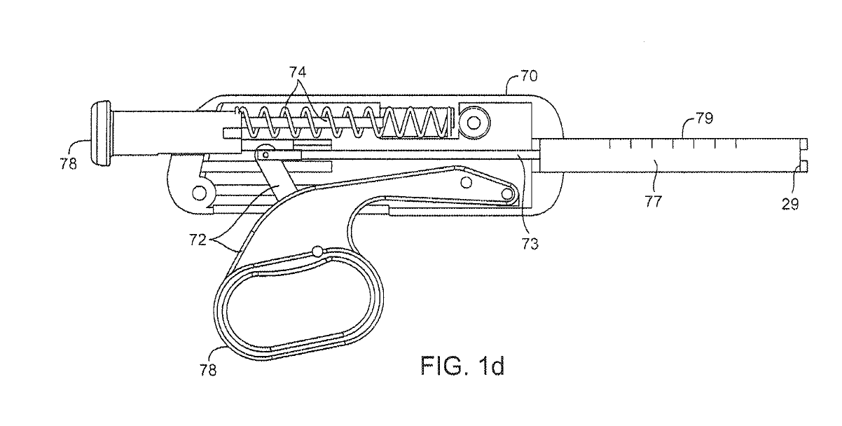

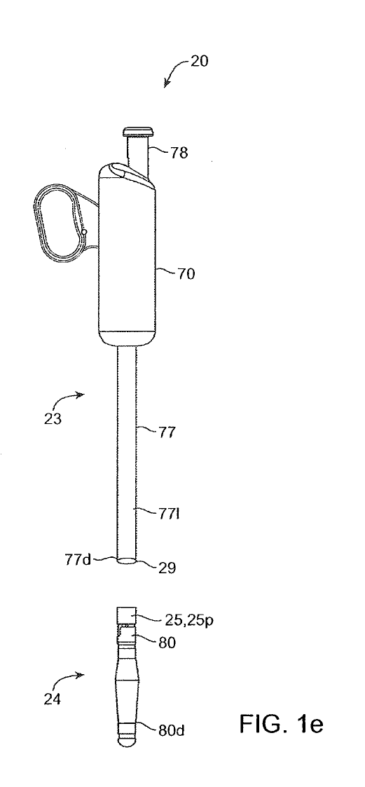

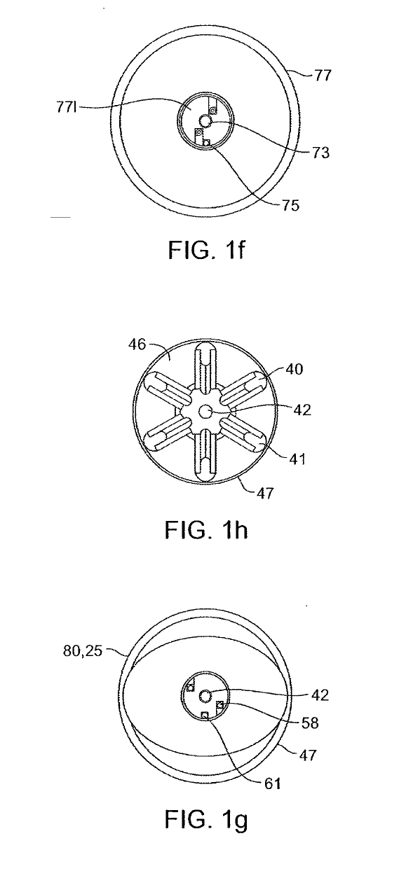

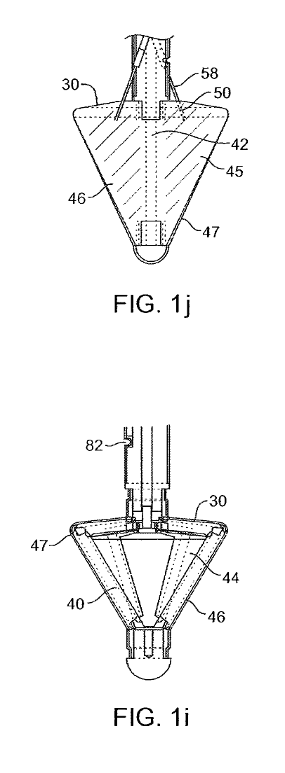

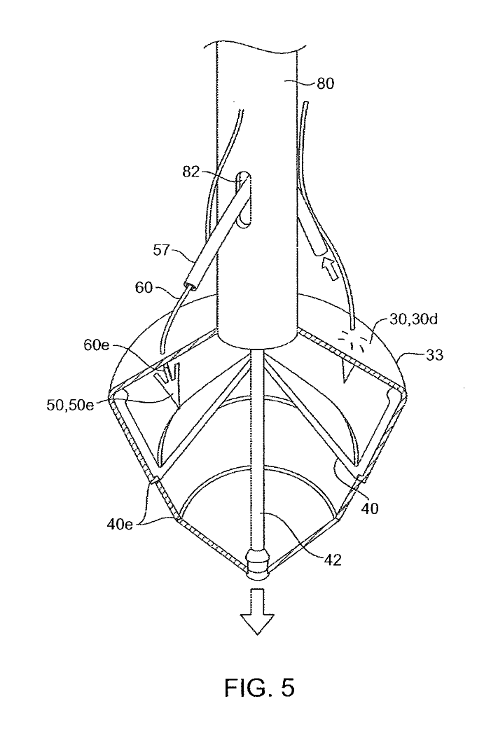

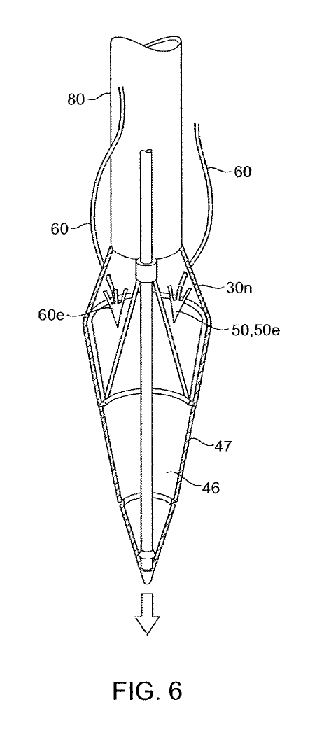

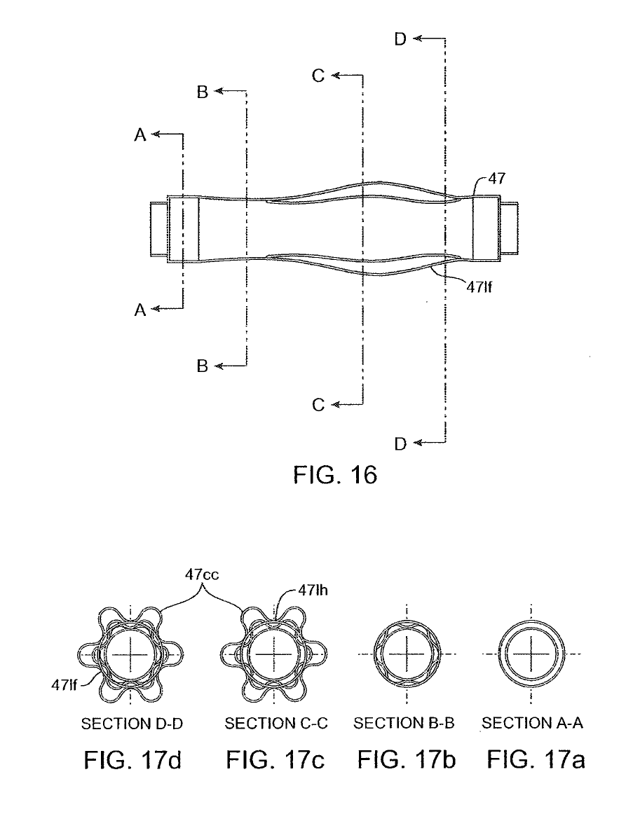

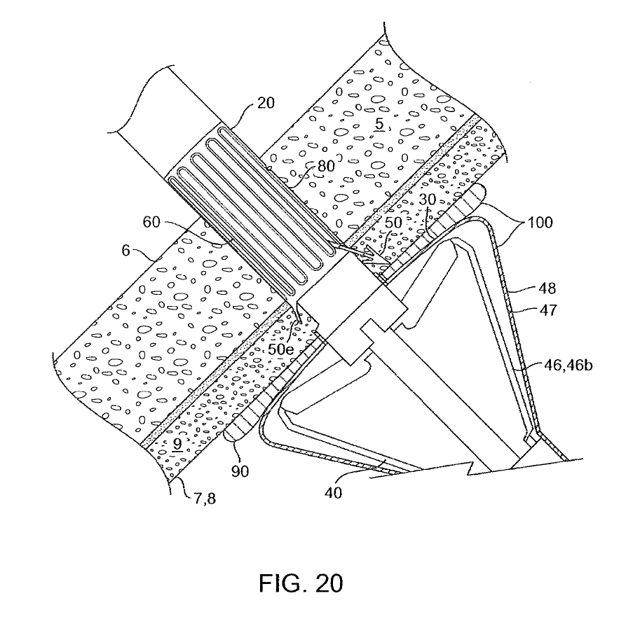

Embodiments of the invention provide apparatus and methods of closing tissue penetration sites made using a trocar or like device during a minimally invasive or other surgical procedure. Referring now to FIGS. 1-20, an embodiment of an apparatus 20 for suturing a tissue penetration site will now be described. Apparatus 20, also known as closure apparatus 20 typically comprises a shaft 25, a suture capture surface 30 coupled to the shaft and at least one pair of penetrating members 50 advanceable from the shaft. Shaft 25 typically includes a suture cartridge 80 that contains one or more sutures 60 that are attached to the penetrating members and used to suture the penetration site. Also, the apparatus will typically include or be configured to be coupled to a hand-piece 70 which may contain mechanisms for advancing the penetrating members and deploying the capture surface as is described herein.

Embodiments of apparatus 20 are particularly useful in suturing a tissue penetration site 10 in an abdominal 5 (having an anterior side 6 and a posterior side 7) or other tissue wall 5. Also, generally, though not necessarily, embodiments of the apparatus and methods are intended for closure of non-vascular tissue penetrations and defects. Such non-vascular tissue penetrations exclude penetrations into blood vessels made for purposes of vascular access by a catheter such as those made in the femoral or brachial arteries. Such non-vascular penetrations and defects include penetrations and defects within the abdominal wall, the thoracic wall, as well as those within various organs including the heart, the atrial-septal, ventricular septal, patent foramen ovale and like defects and penetrations. They also include penetrations and defects within various body cavity walls including the vaginal wall, the cervical wall, the large and small intestinal wall, the stomach wall, the esophageal wall, the sinus walls and like anatomical structures.

The penetrating members are configured to advance one or more tissue penetrating members 50 through tissue and into the capture surface 30 to capture a pair of sutured ends 60e within the surface. The suture ends can be on the same or different sutures. Typically, the apparatus will be configured to advance at least one pair of penetrating members 60 such as needles, through tissue and into the capture surface. However, the apparatus can also be configured to utilize a single needle to advance and capture a first suture end in the capture surface, and then use that same needle to advance and capture a second suture end in the capture surface. The surface can be rotated between each advancement to position the first suture end at a first location and the second suture end at a second location, for example, at a 180° radial offset from the first location. The single penetrating member can carry both suture ends 60e, but place them one at a time (e.g., one for each tissue penetration) or the penetrating member can be configured to pick up a new suture end after each surface penetration. This latter method can be achieved through use of a reciprocating reloading mechanism known in the art. The former approach can be achieved by configuring the penetrating member as a dispenser of suture ends with each suture end having an adhesive or other self-capturing portion 60a that binds to the capture surface when inserted by penetrating member. The suture ends can be vertically or otherwise stacked on the penetrating member in such a way that only the top most suture end is captured in the captured surface. In this way, the penetrating members are able to insert/dispense suture ends with each advancement into the suture capture surface.

In many embodiments, apparatus 20 will have a modular construction including a re-usable portion 23 and interchangeable or disposable portion 24. Reusable portion 23 will typically comprise hand-piece 70 and a section of hand-piece shaft 77. Disposable portion 24 typically comprises cartridge 80 with a coupled deployable frame 40 or other capture member 46. Reusable portion 23 and disposable portion 24 are desirably coupled through a detachable coupling 29 such as a spring loaded, cam lock or quick release coupling known in the art. Typically, coupling 29 will couple the distal end 77d of shaft 77 to proximal end 25p of shaft 25 and/or cartridge 80. However, other juncture points are also contemplated. In use, the detachable coupling allows the surgeon to rapidly detach a spent cartridge 80 and attach a new cartridge 80 having a desired suture type, size, etc. It also reduces the cost to the end user by being able to reuse portions of the apparatus. To that end, hand-piece 70 can be constructed from materials that are readily autoclavable or sterilizable by other sterilization methods available to hospitals. The disposable portion 24 can be fabricated from various medical polymers known in the art which can sterilized by e-beam, plasma and other sterilization methods known in the art. In other embodiments, the disposable portion 24 can also be configured to be cleaned, reloaded with new suture and autoclaved for re-use. Re-usable portion 23 and disposable portion 24 will typically be configured to be packaged separately but can also be packaged together as a kit. Also desirably, re-usable portion 23 is configured to mate with all varieties of disposable portion 24, but in particular embodiments can be configured to mate only with certain disposable portions, such as those configured for pediatric or intrauterine applications. Further as described herein, one or both of the re-usable portion 23 or disposable portion 24 can have a bar-code or other identifying indicia to assure a proper match of the two portions.

Capture surface 30 has a deployed configuration 30d and a non-deployed configuration 30n. The surface is configured to capture a needle and attached suture in the deployed configuration and retain the needle and suture in the non-deployed configuration. The surface is penetrable to allow penetration by a needle or other penetrating member 50 and is also configured to capture the penetrating member 50 and attached suture end 60e either in or beneath surface 30. For example, penetrating member 50 may carry a barb or other element which can pass through the surface but which will be trapped by the surface to prevent withdrawal of the suture when the apparatus is withdrawn from the tissue penetration site. Surface 30 will typically comprise a conformable material such as a mesh or elastic membrane such as silicone, polyurethane or other elastomer known in the art. Suitable meshes include DACRON and other polyesters, polyethylenes, fluoropolymers and other biocompatible polymers known in the art. The surface can be sufficiently conformable to be stretched over an expandable framed as is described below. Also, portions of the surface be constructed of different materials, for example portions of the surface intended for penetration by needle 50, such as target zones 36, can be constructed from more penetrable softer materials (e.g. lower durometer) while the remainder portions can be constructed from harder material more resistant to penetration (e.g., higher durometer).

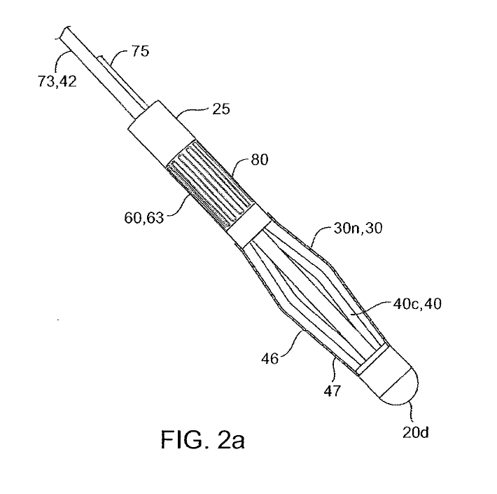

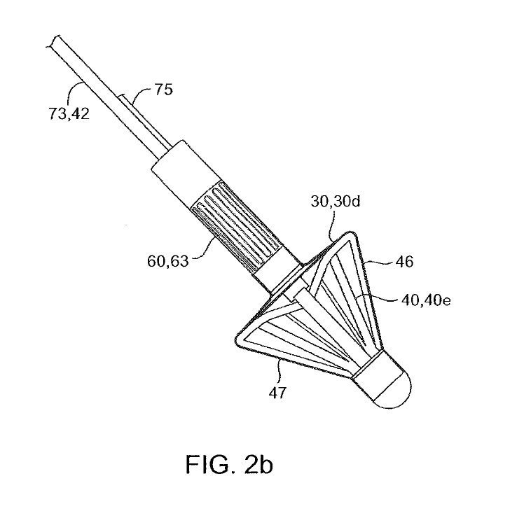

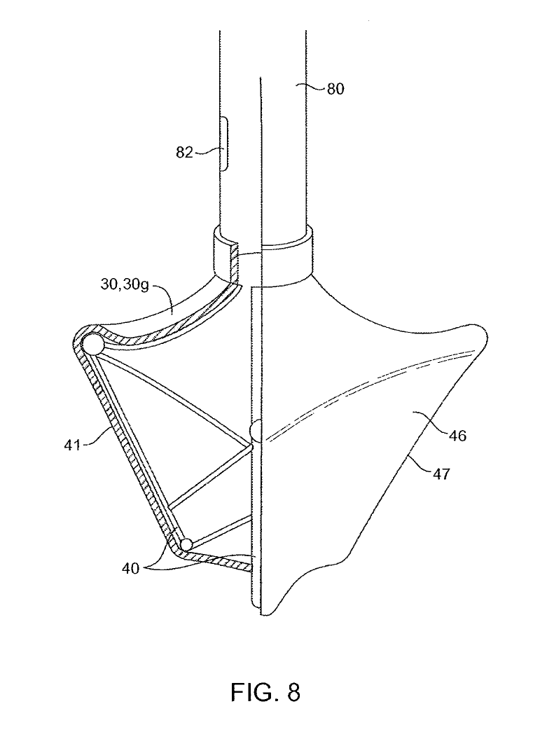

In many embodiments, surface 30 will be mounted or otherwise formed over an expandable frame 40 which can be shifted between a low profile radially constrained configuration 40c and a radially expanded configuration 40e so as to expand capture surface 30 to its deployed configuration as is shown in FIGS. 2a and 2b. Typically the entire frame will be covered by the material forming surface 30 so as to form a protective shroud or sleeve 47 (described below) with surface 30 comprising a portion of shroud 47. The frame will typically have an umbrella or like shape 40u when in the expanded state but other shapes are also contemplated such as a four point frame shown in FIG. 8. The frame can be constructed from one or more frame members or 41 which can in turn be fabricated from various flexible metals or polymers known in the art. For self expanding embodiments, members 41 can be constructed from various shape memory materials such as NITINOL or spring steel. Typically the frame will be configured to be mechanically expanded by means of a deployment member 42 which can be a push pull rod or other mechanical link. Member 42 can be mechanically coupled to a frame deployment mechanism 72 in the hand-piece 70 as is described herein. Alternatively, frame 40 may be expanded by an expandable balloon 44 as is shown in FIG. 1i, or other expandable structure 44 such as a spring based structure or shape memory structure. The frame can also be configured to be self-expanding by means of a shape memory material, in such embodiments, member 42/mechanism 72 can be configured to release the frame from its constrained state wherein it self expands and then pull it back to its constrained state. In still other embodiments, the surface can be deployed without a frame but rather an expandable foams support 45 or other expandable supporting member as is shown in FIG. 1j. Support 45 can be constructed from various memory foams known in the art.

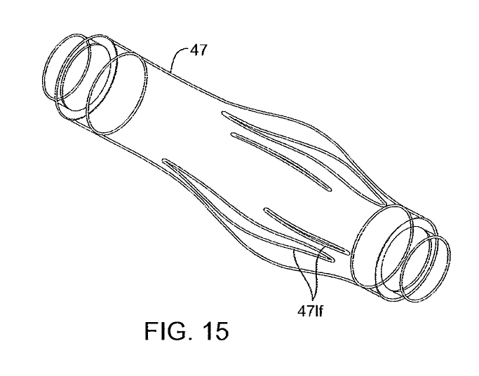

Shroud 47 will typically comprise the same materials as surface 30 and can be formed by various polymer processing methods known in the art (e.g., extrusion, molding, balloon molding and like methods). The shroud can also be pre-shaped or formed to have a particular shape both in the non-deployed and in the deployed state. In preferred embodiments, the shroud can include one or more of longitudinal folds 47lf so as to have a convoluted radial cross-sectional profile 47cc as is shown in FIGS. 15-17. The convoluted shape allows for ease of packing of the shroud in the non-deployed state and a larger surface area in the deployed state with less required deployment force and reduced risk of tearing of the shroud by having a larger deployed circumference of the shroud.

In various embodiments, surface 30 and structure 46 can have various mechanical and material properties to facilitate needle/suture capture and suture placement at the penetration site. For example, the capture surface will desirably have sufficient mechanical rigidity to support the tissue layers overlying the surface such that the penetrating members will readily penetrate through the tissue and the surface without difficulty due to deflection of either the tissue or surface. Also, the surface can have sufficient texture (e.g., from use of a mesh) or adhesive quality to prevent the overlying tissue layers from laterally slipping (e.g., sliding side to side) due to any lateral forces exerted by the needle during needle advancement. Such embodiments thus provide a means of sub-dermal tissue support and tissue stabilization which serve to improve one or more of the accuracy, reliability and reproducibility of needle and suture placement.

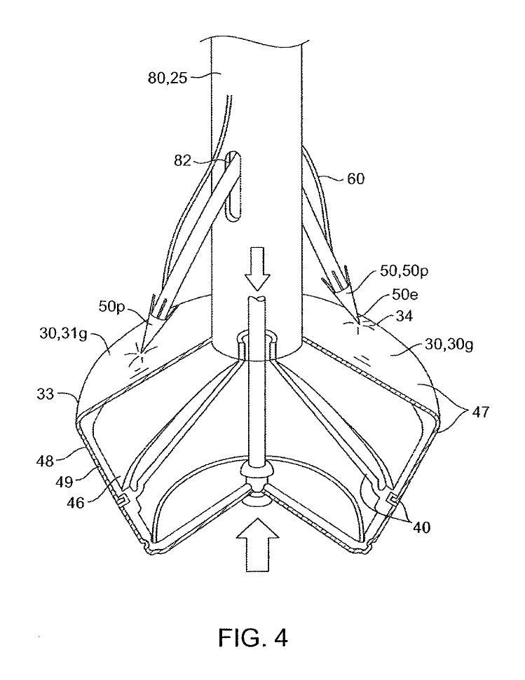

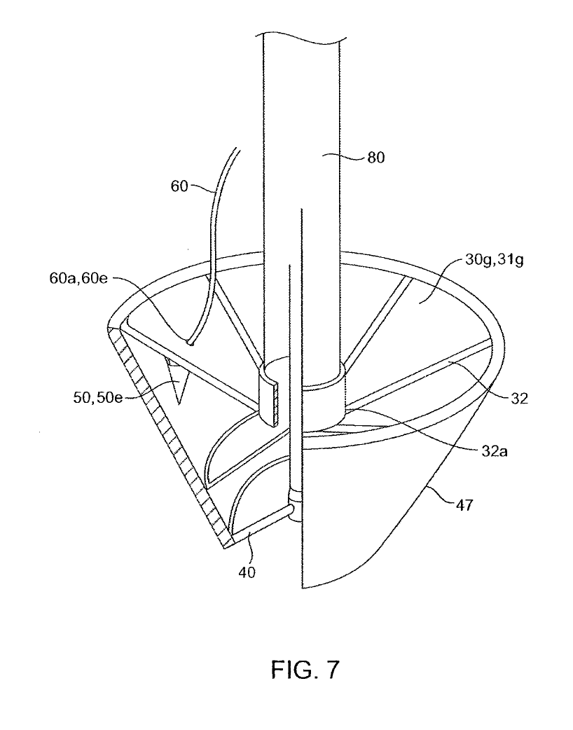

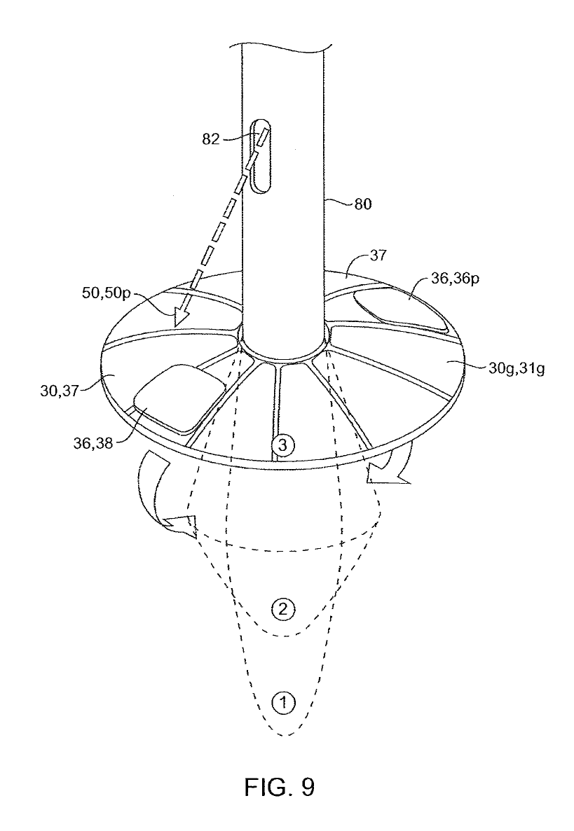

In various embodiments, surface 30 can have a variety of shapes or geometries 30g and orientations. This can be achieved both by the shape of frame 40, the preformed shape of the surface, its positioning relative to shaft 25 and the amount of deployment. In preferred embodiments, the surface has an annular geometry 31g which is generally symmetrical or concentric about shaft 25 as is shown in FIG. 9. However, eccentric and non-annular geometries are also contemplated. The surface can also have a concave 32 or convex profile 33. For example as shown in FIG. 7, the surface can have a conical/concave profile 32 with cone apex 32a facing distal direction. As shown in FIG. 4, in other embodiments surface 30 can have a convex profile 33.

In various embodiments, the whole capture surface can be configured for needle/needle suture capture. Further, such embodiments allow for needle capture into surface 30 independent of an entry point 34 into the surface. Such embodiments can include conical/concave shaped surfaces such as that shown in FIG. 7. Other embodiments of surface 30 allow for needle capture independent of entry point including generally annular shaped surfaces such as that shown in FIG. 9. In use, such embodiments facilitate the tissue penetration closure procedure by allowing the surgeon to perform a suture capture without having to have the capture surface in a precise position or orientation with respect to the penetration site. Also, it allows the surgeon to readily reposition the surface during the course of a closure procedure. For example, the surgeon can place one or more sutures in the surface when its is in a first position and then rotate the surface to a second position and place one or more sutures in the second position. In one embodiment, the surgeon could thus use the apparatus to place a first fastened suture loop at the penetration site and then place a second fastened loop at a 90° or other radial offset from the first loop.

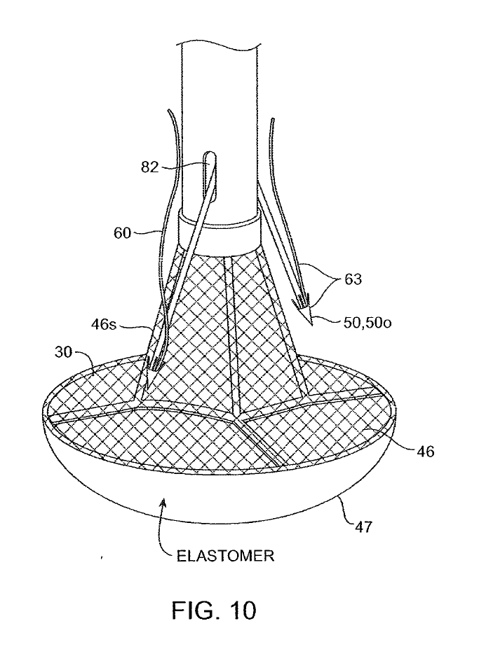

In various embodiments, capture structure 46 can also have a variety of shapes or geometries. This can be achieved both by the structure of framing 40 as well as the preformed shape and material characteristic of the covering shroud 47. In many embodiments, the capture structure will have generally conical and/or umbrella shapes as shown in FIGS. 1-3. It can also include a tapered cylinder or lamp shade-shape as shown in FIG. 7, as well as a mushroom shape shown in FIG. 10. In this latter embodiment, structure 46 can include a stalk portion 46s configured to pre-tension the tissue layers of the penetration site, prior to needle entry and suture placement. In use, the pre-tensioning stalk portion 46s serves to increase the consistency of the suture placement.

As described above, in various embodiments, surface 30 can be configured to have needle or other penetrating member 50 enter at any point in the surface. In preferred embodiments, surface 30 can have at least one target zone 36 which is configured to align with and capture advancing needle 50 as is shown in FIG. 9. Preferably zones 36 include a pair of zones 36p which can be symmetrically disposed on either side of shaft 25 (i.e., they are positioned approximately 180° apart) so as to align with needle pair 50p. Zone 36 can be configured to have particular material and dimensional properties to facilitate entry and capture of the needle in the zone. For example, zone 36 can comprise a mesh or other material 38 that more readily allows entry of the needle through the surface than material in the remainder portion 37 of the surface.

In embodiments where surface 30 is disposed over frame 40 it forms a suture capture structure 46. Suture capture structure 46 can be configured to perform a number of functions. First, as described above, it serves to capture penetrating member 50 along with suture end 60e for the suturing and closure of a tissue penetration site 10. Also, through the use of protective shroud 47, it provides a means of protecting internal organs and other non-target tissue 19 (e.g., blood vessels, nerves, etc.) during placement or tissue penetrating members. Structure 46/shroud 47 performs these functions in a number of ways. First, by serving as a barrier 46b to push away any non-target tissue such as internal organs which may encroach into the space between the capture surface and the peritoneum or other body cavity surface layer 8. Second, by serving as a landing pad or pin cushion for the advancing needles or other penetrating members 50 to prevent them from contacting non target tissue. Third, by preventing the captured needles from exiting the shroud once captured.

The first function can be achieved by sizing and shaping structure 46 to push away encroaching tissue. Suitable shapes can include conical, cylindrical, and pyramidal and like shapes. The latter two functions can be accomplished by configuring the capture surface 30 and shroud wall 48 to have sufficient thickness and hardness to capture the needles in the shroud wall and/or prevent the needles from readily poking through the shroud wall once captured in the shroud wall or the interior 49 of the shroud. In particular, the shroud wall can be configured (e.g. thickness and hardness) to allow penetration by the needles into the shroud when they are advanced using force applied push rods 59 or other needle advancement means, but prevent penetration of the shroud once the needles are captured inside. The protective function of shroud 47 can be further enhanced through the use of one or more secondary capture surfaces 35 positioned within shroud interior 49. In these and similar embodiments, structure 46 can have a baffled construction allowing needles 50 to pass through multiple capture surfaces. In another aspect, structure 46 and shroud 47 also provide means and methods for preventing any non-target tissue from becoming trapped or otherwise encroaching into the space between the abdominal wall and the tissue penetration site. The structure and shroud can be used as a barrier to prevent tissue from entering the tissue penetration site or push out tissue that has entered. In another method of use, the structure can be used to pull up on the peritoneum to make sure that any internal organs or other tissue are not caught within the penetration site and once released, the shroud keeps any tissue from re-entering into the site.

Capture structure 46 can be configured to provide the surgeon with an indication that the apparatus is in good contact with the peritoneum 8 or other inner surface of a selected tissue cavity. This can be accomplished by pulling back on the shaft until it is apparent that the capture surface is in contact with the intra abdominal wall. A simple method of verification is to feel the resistance as the surgeon pulls up on the hand-piece and/or observe that the outer abdomen tracks the upward movement of the apparatus. This has the result of bringing the layers of tissue in intimate contact with the capture surface at the point where the penetrating member(s) exits the shaft and thus securing an adequate “bite” of tissue for placement of a suture. This approach of verification eliminates the need for intra abdominal visualization and/or imaging, and also improves the consistency of suturing since the placement of the capture surface with the abdominal wall is reproducible. This technique can be facilitated by constructing frame 40/structure 47 to have sufficient rigidity to be able to deflect the abdominal wall (or other tissue layer) when pulled against the wall by shaft 25.

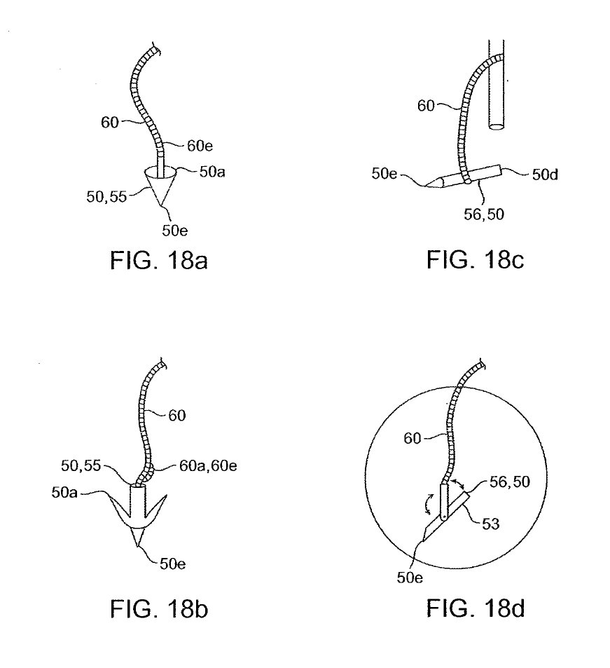

A discussion will now be presented on penetrating members 50. Penetrating member 50 is configured to penetrate tissue at the tissue penetration site as well as capture surface 30. Member 50 can comprise any configuration that is tissue penetrating, including a needle 50. Desirably needle 50 is also configured as an anchor needle 55 which is configured to anchor itself in beneath surface 30. Several embodiments of anchoring penetrating members are shown in FIG. 18a-18d. As shown in 18a and 18b member 50 can have a harpoon or grapple hook shape (with two or more hooks, a particular embodiment can have a tripod shape) that has both a pointed end 50e, as well as an anchoring or retaining feature 50a that serves to hold the penetrating member once it has entered surface 30. FIG. 18c illustrates a T-type anchor with pointed end 50e. Once the pointed end enters the capture surface, the anchor re-orients itself to yield a segment parallel against the capture surface. The T-type anchor is thus a self-orienting anchor 56. FIG. 18d illustrates an embodiment of a penetrating member having a pivotal or other movable portion 53. The movable portion re-orients upon entry into the suture capture surface to anchor the penetrating member into the surface by yielding a segment parallel to the surface or otherwise becoming lodged in or against the surface. Movable portion 53 can also include bendable portions and be moved by an external magnetic force or through the use of a micro-mechanism such as a mems device.

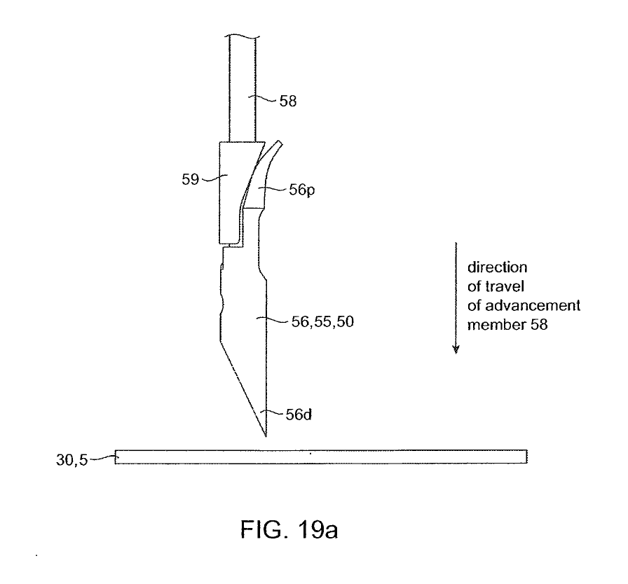

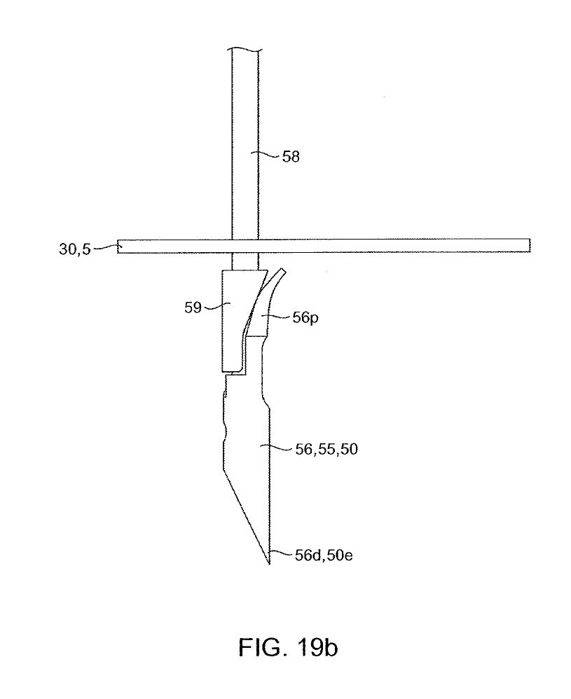

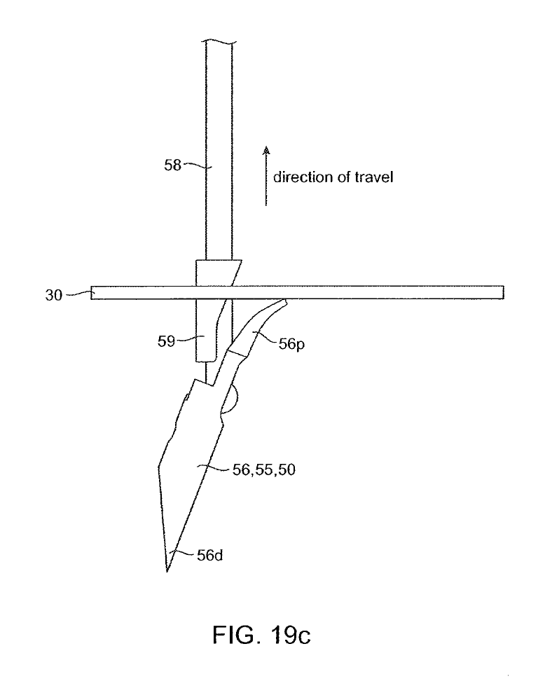

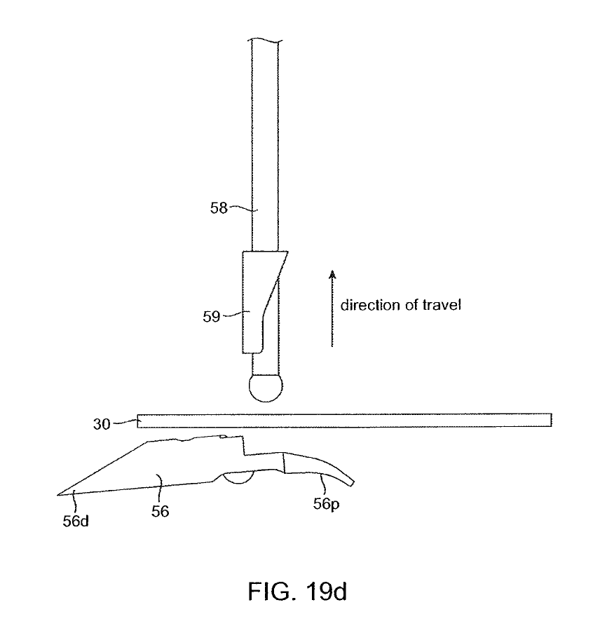

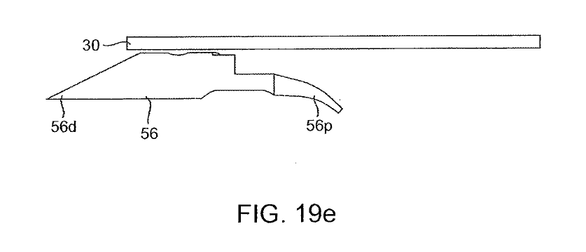

As described above, various embodiments of penetrating members can be a self-orienting anchor 56 configured to allow for entry and then capture in to the surface. Other embodiments of the self-orienting anchor 56 can include an penetrating distal portion 56d which is generally straight and a re-orienting proximal portion 56p. The re-orienting proximal portion 56p can be curved or otherwise shaped to press against the internal portion of the capture surface to change the orientation of member 56 (upon entry into the surface) to a parallel or other orientation which lodges and thus anchors the member in or against the surface. In many embodiments, the proximal portion can be a curved portion which causes the penetrating member to flip from a perpendicular to a generally parallel orientation with respect to the surface when the member is pushed through the surface and the proximal portion contacts the interior of the surface. FIGS. 19a-19e pictorially illustrate the use of such a self-orienting anchor 56, including the position of the penetrating member during the various stages of member deployment into the surface. As shown in the figures, a perpendicular orientation for member 50 during surface entry can be facilitated by use of a support 59 on the end of needle advancement member 58. The contour of the support 59 can mirror the contour of curved proximal portion 56p so that the two components fit together during advancement of member 50 through the capture surface. When the advancement member is withdrawn, the proximal curved portion is no longer supported and now pushes against the surface interior to re-orient the entire member 50 to a substantially parallel orientation with respect to surface 30. This can be facilitated by a slight pulling or tensile force exerted by the attached suture 60 which has a portion still within cartridge 80.

In various embodiments, hand-piece 70 is configured to be held in the hand of the user and will typically include mechanisms 72 for deployment of surface 30 (e.g. by expansion of frame 40) and mechanism 74 advancement of penetrating members 50. Mechanisms 72 and 74 can comprise various spring loaded or cam driven mechanisms known in the art. Also, they typically will each be configured to be coupled to a mechanical linkage. For example, mechanism 72 can be coupled to a push pull rod 73 for deployment of surface 30. Similarly, mechanism 74 can be coupled to a needle driving wire/rod 75. Linkages 73 and 74 can be continuous with corresponding members 42 and 58 or they can be configured to be detachably coupled (e.g. by a cam lock) at coupling 29 or other locations on the apparatus. Typically, linkages 73 and 75 will be contained in the lumen 77l of a shaft 77 that is attached to the distal portion of the hand-piece 70. Shaft 77 will typically be configured to be detachably coupled to shaft 25 at coupling 29, as is described herein. One or both of shafts 77 and 25 can include markings 79 or other indicia to indicate depth of insertion of the apparatus into the tissue penetration site. In use, these markings provide the surgeon with the ability to more accurately position the apparatus and deploy the capture surface in the target penetration site.

Mechanisms 72 and 74 can be independently actuated through the use of actuators 78 such a movable bolts, buttons, levers, triggers, cams, slides and the like. The hand-piece and actuators can be configured to allow the surgeon to actuate each mechanism with a separate finger so that the surgeon can both deploy the surface and advance the penetrating members using only a single hand and without having to change their hand position on the hand-piece. Also, the actuators for either mechanism can be indexed (e.g. between partially and fully deployed positions) and can also be configured to be coupled to a servo control mechanism or the end effector of a surgical robotic device known in the art. In addition to actuators, the hand-piece 70 can also include ports (not shown) for aspiration, fluid delivery, imaging/visualization probes/devices and power couplings.

In many embodiments, cartridge 80 is replaceable and is configured to be detachably coupled to the hand-piece 70 or shaft of re-usable portion 23. The cartridge typically comprises all or a portion of shaft 25 or is otherwise coupled to shaft. The length and width of the cartridge can be standardized or can be sized for the particular surgical application, e.g. shorter cartridge can be used for pediatric applications. The cartridge will typically contain one or more sutures 60 with coupled needles or other penetrating members 50 (which form suture assemblies 63). Suture 60 can have a needle at one or both suture ends 60e. (When the suture contains needles at both ends, a pair 50p of penetrating members are advanced into the tissue with a common length of suture as is described herein). In many embodiments, the cartridge will be packed with multiple suture assemblies 63, for example at least two, or at least three assemblies. Also, the cartridge can be packed with different types of sutures, different lengths etc. In some embodiments, the suture assembly can comprise a single needle having multiple detachably coupled sutures, with each suture having an adhesive or other anchoring portion 60a as is described herein.

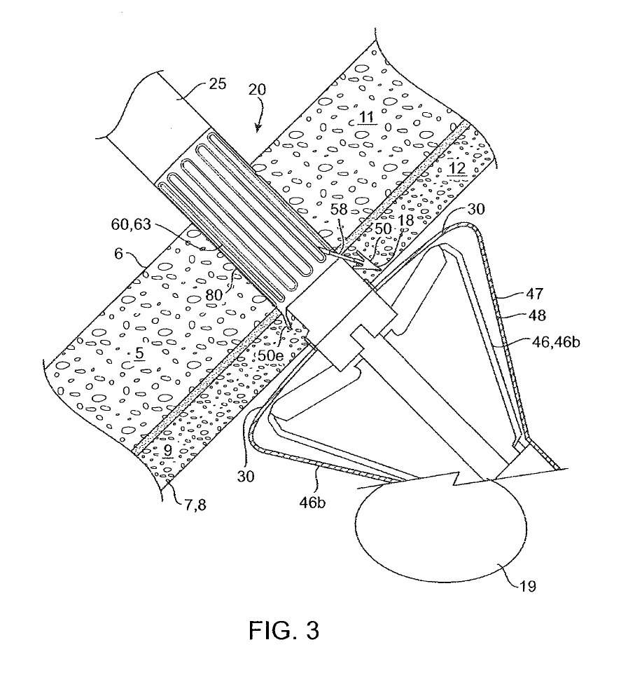

Penetrating members 50 and attached sutures 60 exits in the cartridge through needle exit ports 82 positioned on shaft 25 as is shown in FIG. 3. Ports 82 can also be continuous with an internal guide tube 61 used to guide the attached sutures out of the cartridge as is shown in FIG. 1g. The penetrating member will typically be advanced through the use of one or more needle advancement members 58 which can be contained within the cartridge or can be advanced into it from shaft 25. Advancement members will typically comprise one or more push rods 58 and can be coupled to needle deployment mechanism 74 via mechanical linkage 75. Push rods 58 can be sized to advance needle 50 to a selected distance out of the cartridge and into tissue and the capture surface. Also, push rod 58 need not be advanced into the capture surface. Rods 58 can also be set so that they extend only a set distance to deploy the penetrating members.

In various embodiments, apparatus 20 can be can be configured to be adjusted to set needles or other penetrating members 50 at selectable depths so as to customize the position of the needles based on the location for the tissue penetration site. The height adjustment can be made through adjustment of a range selector (not shown) positioned or coupled to the hand-piece 70, or on cartridge 80. In one embodiment, the range selector that adjusts the point at which the push rods 58 or other advancement member 58 exit shaft 25. The range selector can move both the push rods 58 along with the suture cartridge 80.

Using the range selector or other depth control means, the depth of needle insertion into the walls of the penetration site can be adjustable from at a maximum depth that shroud extends into the penetration site to a minimum depth of just a few millimeters. Once the height adjustment has been set, the apparatus can then be activated to deploy the penetrating members to the desired depth.

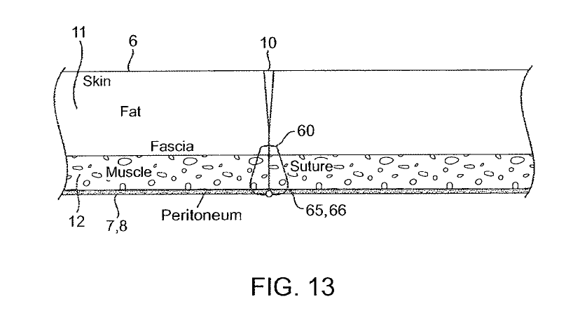

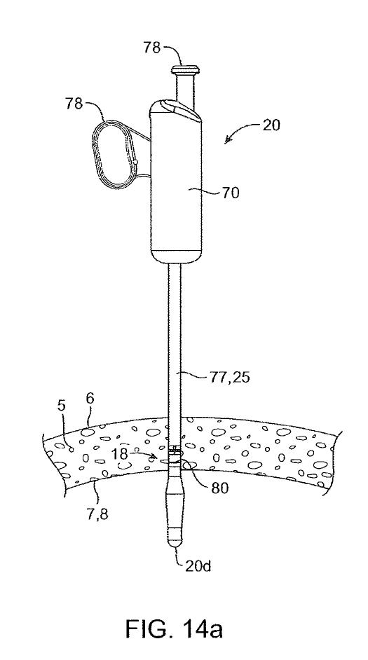

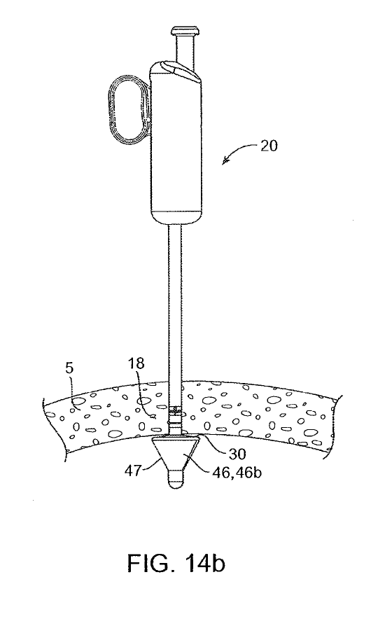

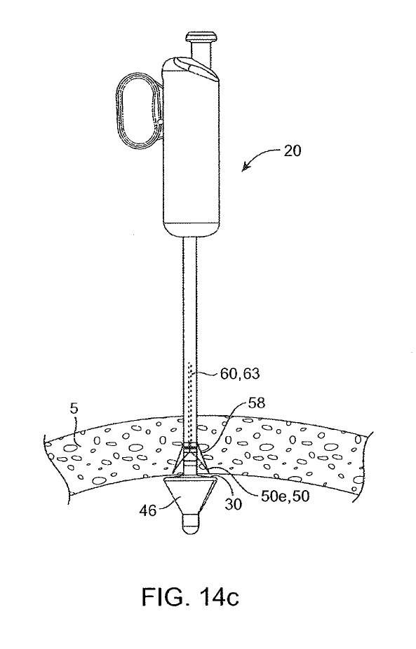

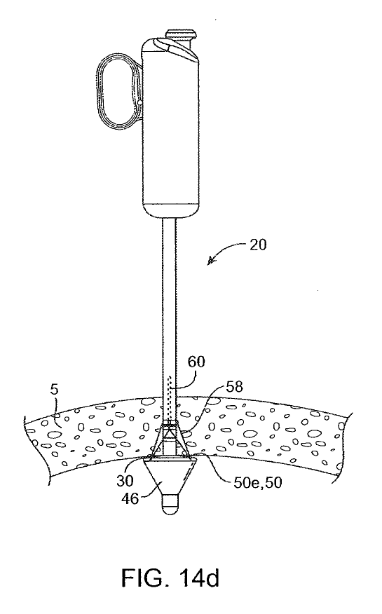

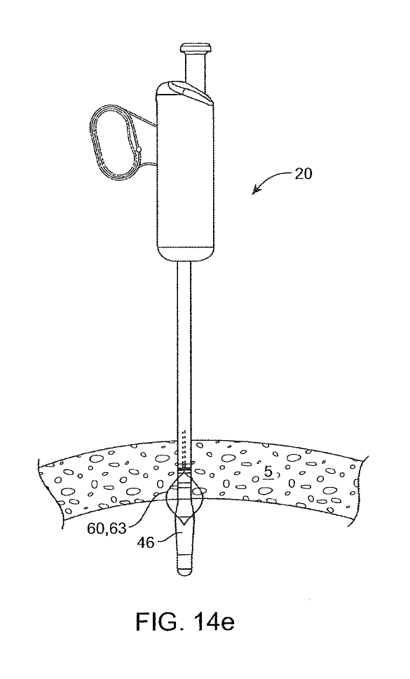

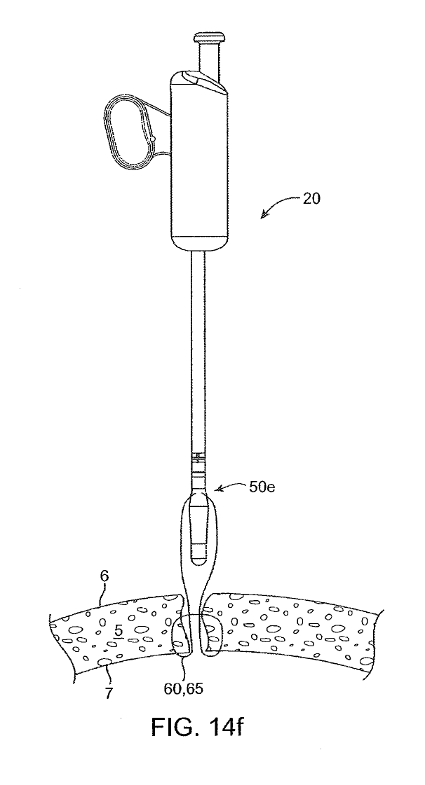

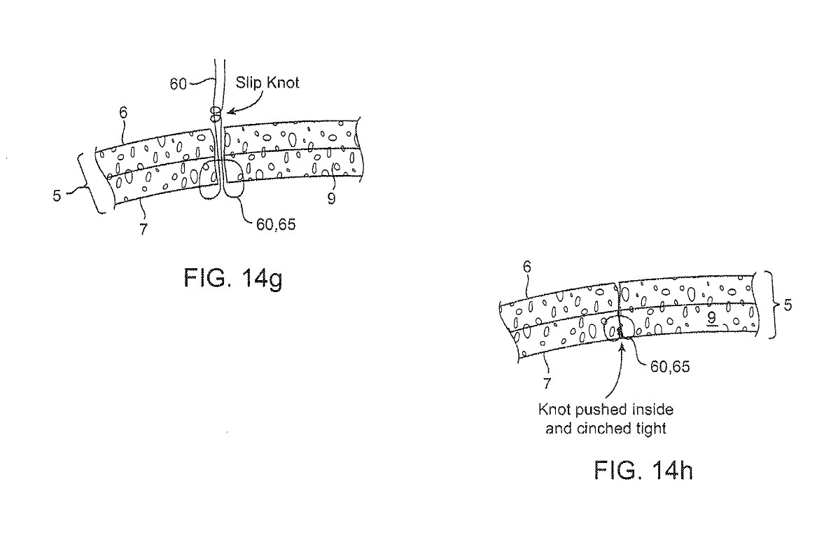

Referring now to FIG. 13 and FIGS. 14a-h, an exemplary embodiment of a method of using the closure apparatus 20 to close a tissue penetration site will now be described. Using hand-piece 70 apparatus which is advanced into the tissue penetration site 10 in the abdominal or other tissue wall 5 while in the non-deployed state. Next, the surface is put in the deployed state and the apparatus is pulled back slightly to position the surface 30 against the posterior side 7 of the site (positioning can be verified by feeling resistance and/or watching the abdominal wall move when the apparatus is pulled). The surgeon could have previously set the needle penetration depth or may do so now using a range selector positioned on the hand-piece. The depth can be used to select a target position 18 for needle entry. Then, the surgeon advances the needles into targeted tissue and into the capture surface 30 where they are captured along with suture 60 in surface 30/capture structure 46. The surgeon then puts the surface in the non-deployed state and withdraws the apparatus out of the penetration site with the suture ends still captured in structure 60, another two portions of the suture exposed in the air and another portion left in a double looped configuration within the layers of tissue at the tissue penetration site. If a surgical cannula (or other access port) was left in place, it is desirably removed simultaneously or near simultaneously with the apparatus. The two exposed portions of suture are then cut away from the capture surface and a slip knot is tied around one of the lengths of exposed suture and then pushed posteriorly down into the penetration site (this can be done using a suture pushing apparatus known in the art) to produce a cinched knot at the posterior side of the penetration site which serves to produce a closed loop 66 of tightened suture which closes the tissue layers 9 (e.g., fascia) on the interior (posterior) side of the tissue penetration site, as is shown FIG. 14h and also in FIG. 13. Also, if the surgeon wishes to close another penetration site at this point, he need only remove and replace the cartridge. This allows for multiple closures to be quickly done without unpacking and reloading a new apparatus for each closure, reducing both procedure time and cost.

Once the interior tissue layers have been closed and the apparatus has been removed from the tissue penetration site, the physician need only to place a simple stitch at the surface layer of the skin to complete the closure procedure. This approach allows for faster closing of the penetration site and improved healing of the site with fewer post-surgical complications including infection. In particular, by closing the penetration site on the interior side of the tissue (vs. the exterior side), the risk of post surgical herneation into the penetration site is reduced because tissue can not readily be forced or otherwise migrate into the penetration site.

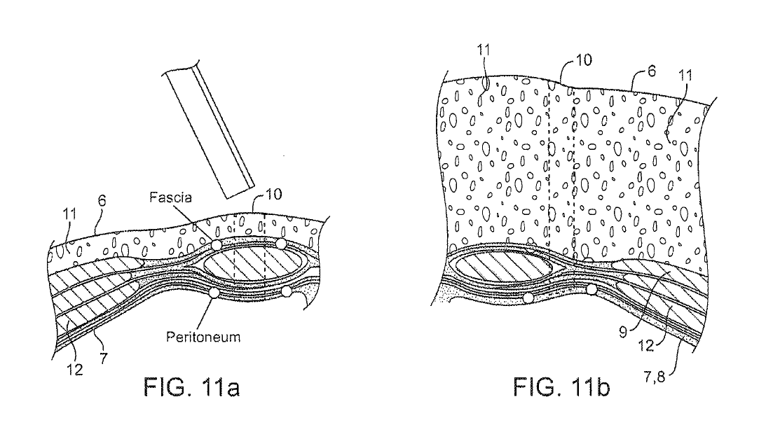

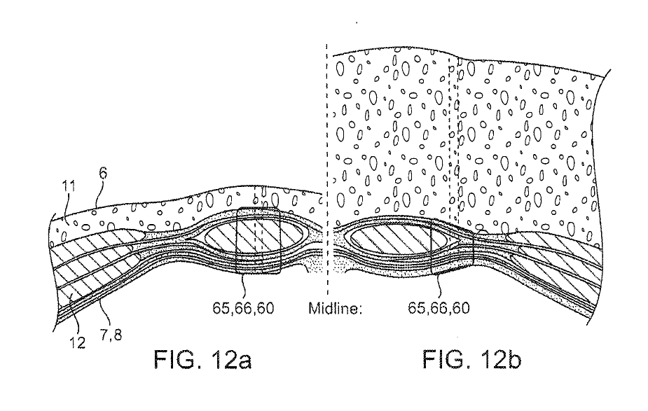

As discussed herein, embodiments of the apparatus provide a number of means of controlling the placement, depth and positioning of sutures 60 in the penetration site 10. These include a needle depth range selector, depth indicia on the apparatus shaft, as well as the technique of pulling the apparatus upwardly to assure contact of the capture surface with the peritoneum or other posterior surface layer 8. As shown in FIGS. 11-12, one or more of these means and methods can be used to accurately and reproducibly position a suture 60 at a desired position 18 in a target penetration site 10 independent of the fat content or patient's body mass index. Such methods can be used to reproducibly position the suture 60 in the posterior portion 12 of penetration site 10 so as to produce a suture path 65 which closes the site on the posterior side 7 of the site. Closing the penetration site in this manner eliminates or reduces the incidence of herniation of subjacent organs or other tissues into the site, thus reducing a number of related post-surgical complications (e.g., infection, etc.). Embodiments can also be configured to place the suture in an anterior portion 11 of the site, if so desired.

Referring now to FIG. 21, in various embodiments, apparatus 20 can also be configured to deliver and suture a prosthetic membrane 90 or other structure 90 at or near the tissue penetration site. Apparatus 20 and membrane 90 can comprise a prosthetic structure delivery system 100. The prosthetic membrane can be carried by capture surface 30 by a detachable means such as low strength releasable medical adhesive or other low force releasable attachment means known in the medical arts (e.g., VELCRO). Similar to surface 30, membrane 90 can have a non-deployed and deployed state so that it can be readily passed through tissue penetration site 10. When the surface is put into the deployed state, the membrane is desirably positioned against the posterior side of the peritoneum or other cavity wall. Then needle 50 and attached sutures 60 are advanced both through tissue wall 5 and the membrane 90 before being capture by surface 90. The sutures hold the membrane against the peritoneal layer with sufficient force such that when surface 30 is then put in the non-deployed state the low force adhesive releases the membrane from the surface. Example membranes 90 can include one or more surgical meshes or PTFE membranes known in the surgical arts. The membrane can be shaped and sized to buttress a particular sized tissue penetration. Also, it can positioned at selected locations on surface 30 depending on the application and can cover all or a portion of the surface. The membrane can be pre-attached to surface 30, or the surface and membrane can be configured to allow the surgeon to attach the membrane within the operating theater, for example using VELCRO or other reversible attachment means known in the medical arts. In use, such methods allow the surgeon to select a membrane which best fits the particular penetration site or defect. He or she can even re-size and reattach the membrane to his own liking and attach or re-attach it. This reduces both operating time and cost required in opening a new package of surgical membrane if a particular one does not fit.

Embodiments of system 100 can be used to repair tissue penetrations as well as various anatomical defects including hernias and other defects in the abdominal wall, as well as various uterine defects and defects in various organs including the heart and lung.

Specific embodiments of delivery system 100 can be configured to repair a number of structural defects in the heart, including without limitation, patent foramen ovale (PFO), atrial septal defects (ASD), ventricular septa defects (VSD). Such embodiments can be configured to be introduced percutaneously through an artery in the groin (such as the femoral artery) and the advanced proximally into the selected chamber of the heart (e.g. the atria or ventricles). Accordingly, apparatus 20 can be sized and otherwise configured for such introduction and advancement using angioplasty catheter fabrication techniques and deployment methods known in the art. For example, guiding catheters and guide wires can be used for introduction and positioning purposes. Also, membrane 90 can be sized and other otherwise configured for correction of a particular defect of a particular size, e.g. a PFO having a particular diameter. The size of the defect and can be determined from various coronary imaging methods known in the art. System 100 can include other apparatus known in the minimally invasive surgical arts for cutting and cinching the sutures once advanced into the selected target tissue site. Also, the sutures can be configured with an adhesive self anchoring portion described herein, such that cutting and cinching are not necessarily needed. The self anchoring portion could be configured to anchor within membrane 90 and need not be advanced into the capture surface. Alternatively, the suture capture surface or another portion of the apparatus could include means for cutting and/or cinching the introduced suture.

### CONCLUSION

The foregoing description of various embodiments of the invention has been presented for purposes of illustration and description. It is not intended to limit the invention to the precise forms disclosed. Many modifications, variations and refinements will be apparent to practitioners skilled in the art. For example, embodiments of the closure apparatus and related methods can be configured for performing closures in a number of locations in the body including the abdominal, thoracic and other chest regions, as well as in various organ systems including the heart, GI tract, renal, brain, eye, ear, and other anatomical regions such as the spine, etc. Embodiments of the apparatus can also be sized or otherwise adapted for pediatric and neonatal applications, as well as for intrauterine applications.

Elements, characteristics, or acts from one embodiment can be readily recombined or substituted with one or more elements, characteristics or acts from other embodiments to form numerous additional embodiments within the scope of the invention. Moreover, elements that are shown or described as being combined with other elements, can, in various embodiments, exist as stand alone elements. Hence, the scope of the present invention is not limited to the specifics of the described embodiments, but is instead limited solely by the appended claims.

What is claimed is:

1. A method for suturing a penetration site in non-vascular tissue, the site having a anterior side and a posterior side, the method comprising:
deploying a suture capture surface over a posterior region of the tissue circumscribing at least a portion of the penetration site;
advancing at least one penetrating member in a posterior direction through the tissue to deliver a pair of suture ends into the suture capture surface so as to capture the suture ends;
withdrawing the suture capture surface to bring the captured suture ends to an anterior side of the tissue; and
fastening a suture loop through the penetration site.

2. The method of claim 1, wherein the suture ends are delivered to the suture capture surface by advancing at least one pair of penetrating members into tissue.

3. The method of claim 1, wherein the suture ends are delivered to the suture capture surface by sequentially advancing the at least one penetrating member into a first tissue location and a second tissue location.

4. The method of claim 1, wherein deploying comprises drawing the suture capture surface against the posterior region of tissue in an anterior direction to engage the tissue.

5. The method of claim 4, wherein deploying comprises pulling a capture surface structure support to draw the suture capture surface against the tissue on the posterior side of the tissue penetration site.

6. The method of claim 1, wherein the suture ends are advanced into tissue at locations in the tissue penetration site that are at a selectable depth relative to the anterior side of the tissue.

7. The method of claim 1, wherein the capture surface substantially protects non-target tissue from penetration by the at least one penetrating member.

8. The method of claim 1, wherein the at least one penetrating member comprises a needle, an anchoring member, or an anchoring needle.

9. The method of claim 1, wherein the penetrating member is advanced through the use of an advancement member mechanically associated with the penetrating member.

10. The method of claim 1, wherein the capture surface captures the suture independent of a point of entry into the surface.

11. The method of claim 1, wherein substantially the entire capture surface is configured to capture the suture.

12. The method of claim 1, wherein the capture surface is deployed by expanding an expandable structure.

13. The method of claim 12, wherein the expandable structure comprises a mechanically expandable frame, a balloon, a foam structure, a spring or a shape memory structure.

14. The method of claim 12, wherein the expandable structure pushes away non-target tissue encroaching into the space between the suture capture surface and the posterior side of the tissue penetration site.

15. The method of claim 1, wherein the capture surface is deployed by the application of a hydraulic or pneumatic force.

16. The method of claim 1, wherein the capture surface circumscribes at least 30% of the penetration site.

17. The method of claim 1, wherein the capture surface circumscribes at least 60% of the penetration site.

18. The method of claim 1, wherein the capture surface circumscribes substantially the entire penetration site.

19. The method of claim 1, further comprising:
manipulating the suture to sub-dermally close the tissue penetration site.

20. The method of claim 19, wherein the sub-dermal closure prevents herniation into the penetration site.

21. The method of claim 1, wherein the suture ends are on a common length of suture, wherein fastening comprises:
drawing the suture ends together to close the penetration site; and
fastening the suture together after the penetration site has been closed.

22. The method of claim 1, wherein the suture ends are on two separate lengths of suture, wherein fastening comprises:
attaching the two lengths together;
drawing the two attached lengths to close the penetration site; and
fastening the suture together after the penetration site has been closed.

23. The method of claim 1, wherein the suture ends are on two separate lengths of suture, wherein fastening comprises:
attaching the two lengths together;
exchanging the attached lengths with a continuous length of suture;
drawing the continuous length together to close the penetration site; and
fastening the suture together after the penetration site has been closed.

24. The method of claim 23, wherein at least two pairs of penetrating members are advanced through the tissue.

25. The method of claim 1, wherein the tissue penetration site was made by a trocar, a surgical access device or a laparoscopic access device.

26. The method of claim 1, wherein the tissue penetration site was made for a cannula, having a diameter greater than about 4 mm.

27. The method of claim 1, wherein the tissue penetration site is in an abdominal, thoracic or chest wall.

28. The method of claim 1, wherein the tissue penetration site is in an organ, lung, heart, stomach or an intestinal wall.

29. The method of claim 1, wherein the tissue penetration site is in a dermal, muscular, adipose, cartilage or fascial layer.

30. The method of claim 1, wherein the suture capture surface has an annular geometry substantially symmetric about a shaft.

31. The method of claim 30, wherein the suture capture surface is mounted on a frame which can be shifted between a radially constrained configuration and a radially expanded configuration.

32. The method of claim 31, wherein the frame is expanded by advancing the frame from a constraining receptacle in or on the shaft.

33. The method of claim 32, wherein the frame is expanded by activating a mechanical linkage.

34. The method of claim 32, wherein the frame is expanded by hydraulic or pneumatic means.

35. The method of claim 1, wherein the suture capture surface is asymmetric about a shaft.

36. The method of claim 1, wherein the suture capture surface has a non-annular geometry.

37. The method of claim 1, further comprising:
advancing the penetrating member into the capture surface to anchor the suture to the suture capture surface.

38. The method of claim 37, wherein the penetrating member re-orients upon entry into the capture surface to anchor the suture to the suture capture surface.

39. The method of claim 38, wherein re-orienting comprises pivoting one portion of the penetrating member relative to another portion.

40. The method of claim 1, wherein the capture surface is deployed and the penetrating members are advanced using a single hand.

41. The method of claim 1, further comprising:
advancing the penetrating members through a prosthetic structure; and
manipulating the suture to attach the prosthetic structure to the tissue penetration site.

42. The method of claim 41, wherein the prosthetic structure is carried by the suture capture surface.

43. The method of claim 41, wherein the prosthetic structure is a surgical membrane.

44. The method of claim 41, wherein the prosthetic structure is attached to a posterior side of the tissue penetration site.

45. A method for repairing a defect in a tissue wall, the method comprising:
deploying a suture capture surface and a prosthetic structure over a region of the tissue circumscribing at least a portion of the defect;
advancing at least one penetrating member through the tissue to deliver a pair of suture ends into at least the prosthetic structure so as to capture the suture ends;
withdrawing the suture capture surface; and
fastening a suture loop through the defect.

46. The method of claim 45, wherein the at least one penetrating member is advanced through the suture capture surface to capture the suture ends.

47. The method of claim 45, wherein the capture surface pushes away non-target tissue from the tissue defect.

48. The method of claim 45, wherein the at least one penetrating member comprises a pair of penetrating members.

49. The method of claim 45, wherein the prosthetic structure is a surgical membrane.

50. The method of claim 45, wherein the prosthetic structure is carried by the suture capture surface.

51. The method of claim 45, wherein the defects is an atrial-septal defect, a ventricular-septal defect or a patent foramen ovale.

52. An apparatus for suturing a tissue penetration site, having an anterior and a posterior side, the apparatus comprising:
a shaft;
a suture capture surface coupled to the shaft, the surface having a deployed configuration and a non-deployed configuration, wherein the capture surface is configured to capture a suture in the deployed configuration and retain the suture in the non-deployed configuration; and
at least one penetrating member, wherein each member is configured to be coupled to a suture end, the at least one penetrating member being advanceable from the shaft in a posterior direction relative to the penetration site to deliver the suture end portion to the suture capture surface when the surface is in the deployed configuration.

53. The apparatus of claim 52, wherein the suture ends are delivered to the suture capture surface by advancing at least one pair of penetrating members into tissue.

54. The apparatus of claim 52, wherein the penetrating member is a needle, an anchoring member or an anchoring needle.

55. The apparatus of claim 52, wherein the penetrating member includes a movable portion which re-orients upon entry into the suture capture surface to anchor the penetrating member into the surface.

56. The apparatus of claim 55, wherein the movable portion is a pivotal portion.

57. The apparatus of claim 52, further comprising at least one advancement member advanceable from the shaft, the at least one advancement member configured to advance the at least one penetrating member into tissue.

58. The apparatus of claim 52, wherein the suture capture surface comprises one of a membrane, a mesh membrane, an elastic membrane, an elastomer, a foam, a foam elastomer, silicone or polyurethane.

59. The apparatus of claim 52, wherein the suture capture surface has an inward conical shape.

60. The apparatus of claim 52, wherein the suture capture surface includes at least one target zone configured to align with and capture an advanced penetrating member.

61. The apparatus of claim 60, wherein the at least one target zone includes a pair of target zones configured to align with and capture a pair of penetrating members.

62. The apparatus of claim 61, wherein the pair of target zones are disposed symmetrically on either side of the shaft.

63. The apparatus of claim 60, wherein the target zone comprises a different thickness or material from a remainder portion of the capture surface.

64. The apparatus of claim 52, wherein the surface is deployed by pneumatic or hydraulic deployment means.

65. The apparatus of claim 52, wherein the surface has a convoluted radial cross-sectional shape in the non-deployed configuration.

66. The apparatus of claim 52, further comprising an expandable structure associated with the surface, the expandable structure configured to deploy the surface.

67. The apparatus of claim 66, wherein the expandable structure comprises a balloon, a spring, a shape memory structure or an expandable foam structure.

68. The apparatus of claim 66, wherein the expandable structure is shaped and sized to push away non-targeted tissue encroaching into a space between the suture capture surface and the posterior side of the tissue penetration.

69. The apparatus of claim 52, wherein the suture ends are on a common length of suture, whereby the suture ends may be drawn together to close the penetration site.

70. The apparatus of claim 52, wherein the suture ends are on two separate lengths of suture, whereby the two lengths may be tied together to permit the two ends to close the penetration site.

71. The apparatus of claim 52, wherein the suture capture surface has an annular geometry generally symmetric about the shaft.

72. The apparatus of claim 52, wherein the suture capture surface is mounted on a frame which can be shifted between a radially constrained configuration and a radially expanded configuration.

73. The apparatus of claim 72, wherein the frame has sufficient rigidity to deflect an abdominal or thoracic wall when a pull force is applied to the frame.

74. The apparatus of claim 72, wherein the frame is expanded by advancing the frame from a constraining receptacle in or on the shaft.

75. The apparatus of claim 72, wherein the frame is expanded by actuating a mechanical linkage coupled to the frame.

76. The apparatus of claim 52, further comprising a suture cartridge coupled to the shaft, the cartridge configured to hold at least one suture and a tissue penetrating member.

77. The apparatus of claim 76, wherein the cartridge is integral to the shaft.

78. The apparatus of claim 76, wherein the cartridge is configured to be engaged by at least one advancement member for advancing the at least one penetrating member into tissue.

79. The apparatus of claim 52, wherein the shaft is configured to be coupled to a hand-piece.

80. The apparatus of claim 79, wherein the shaft is configured to reversibly detach from the hand-piece.

81. The apparatus of claim 79, wherein the hand-piece includes a first mechanism for deploying the capture surface.

82. The apparatus of claim 81, wherein the hand-piece includes a second mechanism for advancing the at least one penetrating member.

83. The apparatus of claim 82, wherein the second mechanism is configured to allow the user to select a penetration depth of the at least one penetrating member.

84. The apparatus of claim 79, wherein the hand-piece is configured to be reusable.
